(19) United States
(12) Patent Application Publication
(10) Pub. No.: US 2016/0310332 A1
(43) Pub. Date: Oct. 27, 2016

(54) FASTENING MEMBER FOR HOOK-AND-LOOP FASTENER AND METHOD FOR MANUFACTURING THE SAME, AND SANITARY PRODUCT

(71) Applicant: 3M INNOVATIVE PROPERTIES COMPANY, Saint Paul, MN (US)

(72) Inventor: Kenichiro Morishita, Sagamihara-city (JP)

(21) Appl. No.: 15/106,145

(22) PCT Filed: Dec. 18, 2014
(86) PCT No.: PCT/US2014/071166

§ 371 Date: Jun. 17, 2016

(30) Foreign Application Priority Data
Dec. 20, 2013 (JP) 2013-264423

(51) Int. Cl. A61F 13/62; A61F 13/15; A44B 18/00

(52) U.S. Cl. CPC A61F 13/622; A44B 18/0073; A44B 18/0011; A61F 13/15756

(57) ABSTRACT

A fastening member for a hook-and-loop fastener including a base material layer having a fastening portion and a printed layer directly fixed on the base material layer is provided. A method for manufacturing a fastening member for a hook-and-loop fastener including a printed layer and a base material layer having a fastening portion is also provided, including the steps of forming a printed layer on a liner, transferring the printed layer to a base material layer having a fastening portion, and removing the liner from the printed layer. A sanitary product including the fastening member for a hook-and-loop fastener is also provided.

### TECHNICAL FIELD

This disclosure relates to a fastening member for a hook-and-loop fastener and a method for manufacturing the same, and a sanitary product.

### BACKGROUND

Conventionally, hook-and-loop fasteners are widely used for fixing, binding, and the like various articles such as textile products, plastic products, paper products, industrial parts, electronic parts, and building materials; while, for example, a sanitary product (for example, a paper diaper or the like) with a hook-and-loop fastener attached as a fastening material is known. Hook-and-loop fasteners of various engaging patterns are known, such as a pair of a male member having a hook-shaped engaging element and a female member engageable with the engaging element.

Japanese Unexamined Patent Application Publication (Translation of PCT Application) No. 2008-516742A describes a knitted fabric female fastening portion for a mechanical fastener comprising a knitted fabric having a first surface and a second surface, the first surface being capable of engaging with a male fastening portion, an underlying substrate joined to the second surface of the knitted fabric, and a bonding layer positioned between the knitted fabric and the underlying substrate, the bonding layer bonding together the knitted fabric and the underlying substrate and having a first plurality of non-intersecting bond lines, and a second plurality of non-intersecting bond lines which are combined to form a pattern of intersecting bond lines that defines tessellating pattern elements.

Japanese Unexamined Patent Application Publication No. 2005-270625A describes a joining material for Velcro® fasteners, particularly diaper fasteners, comprising a base sheet (1) and a textile substrate (2) laminated onto the base sheet (1), the textile substrate having loops (4) on the surface, suitable for joining the surface and engaging tooth hooks, wherein the base sheet (1) and the textile substrate (2) are not joined over the entire surface.

Japanese Unexamined Patent Application Publication No. H10-127311A describes a hook-and-loop fastener female member with an adhesive comprising a reinforcing layer stacked on a non-fastener surface of the hook-and-loop fastener female member forming a substrate, and an adhesive layer provided on the surface of the reinforcing layer of the substrate.

Japanese Unexamined Patent Application Publication No. H4-276251A describes an absorbent product comprising a composite material having a supporting body and an absorbent body on this supporting body, the composite material being further provided with front and back end portions oppositely disposed, a first releasably engageable member on the back end portion of the composite material, a second releasably engageable member related to the front end portion of the composite material, the first and second engageable members being releasably engageable with each other, and a buckling means joining the second engageable member to the front end portion for assisting the engagement between the first and second engageable members, whereby, the front end portion tends to buckle or distort during engagement.

Japanese Unexamined Patent Application Publication No. H10-85012A describes a method for manufacturing a laminated assembly having a first layer having a surface fixed by an adhesive to a first surface of a second layer, the second layer having a mesh structure and a loop projecting from the second surface, comprises a step of applying and adhering a thin adhesive film onto the first surface of the second layer under an adhesive amount and viscosity condition controlled so that the applied adhesive film separates itself to form mesh portions and to deform into vertical and/or horizontal beads.

### SUMMARY

Conventionally, an article having a cloth and a hook-and-loop fastener attached to the cloth (for example, a sanitary product such as a diaper), a fastening member having a printed layer may be used as a hook-and-loop fastener. For example, a fastening member for a hook-and-loop fastener having a printed layer can be obtained by adhering a print film obtained by forming the printed layer on a printed layer support film to a non-fastening portion forming side of a base material layer having a fastening portion on at least one of two main surfaces. The base material layer is optically transparent to the extent that the printed layer is visible through the base material layer. Fixing the surface of the printed film side of this type of fastening member for a hook-and-loop fastener to a cloth allows a designed hook-and-loop fastener to be formed. However, it has now been found that such a printed layer support film leads to reduced breathability and flexibility of the article with the fastening member attached, and this reduction in breathability and flexibility is not preferable in applications where the wearing feeling is important such as, for example, a sanitary product. Here, cast polypropylene (CPP) films, biaxially oriented polypropylene (BOPP) films, and the like are given as films that can be used as a printed layer support film, but these films are usually non-breathable, and may lead to reduced breathability. In addition, the cost of raw materials for the printed layer support film and the manufacturing cost of a printing method which uses the printed layer support film cannot be ignored. Therefore, it would be desirable to have a fastening member for a hook-and-loop fastener that avoids the deficiencies described above while still having a printed layer.

Accordingly, among the benefits that may be achieved by the present invention include providing a fastening member for a hook-and-loop fastener that can address one or more of the deficiencies described above, in that it can improve breathability and flexibility while still having a printed layer, and is additionally advantageous in cost; a method for manufacturing the same; and a sanitary product that includes the fastening member for a hook-and-loop fastener as described herein.

In one aspect, the present invention includes a fastening member for a hook-and-loop fastener comprising a base material layer having a fastening portion, and a printed layer directly fixed on the base material layer.

In another aspect, the present invention includes a method for manufacturing a fastening member for a hook-and-loop fastener including a printed layer and a base material layer having a fastening portion, comprising a process of forming a printed layer on a liner, a process of transferring the printed layer to a base material layer having a fastening portion, and a process of removing the liner from the printed layer.

In a further aspect, the present invention includes a sanitary product including the fastening member for a hook-and-loop fastener comprising a base material layer having a fastening portion, and a printed layer directly fixed on the base material layer.

Thus, according to the present invention, a fastening member can be provided for a hook-and-loop fastener that excels in breathability and flexibility while still having a printed layer, and that is additionally advantageous in cost, a method for manufacturing the same, and a sanitary product that includes the fastening member for a hook-and-loop fastener are provided.

### BRIEF DESCRIPTION OF THE DRAWINGS

FIG. 1 is a diagram illustrating an example of a fastening member for a hook-and-loop fastener according to an aspect of the present invention.

FIG. 2 is a diagram illustrating an example of a fastening member for a hook-and-loop fastener according to an aspect of the present invention.

FIG. 3 is a diagram illustrating an example of a method for manufacturing a fastening member for a hook-and-loop fastener according to an aspect of the present invention.

FIG. 4 is a diagram describing a method for measuring the rigidity of a fastening member in the disclosed embodiments.

### DETAILED DESCRIPTION

An exemplary aspect of the present invention will be described hereafter, but the present invention is not limited to the following aspect, and any modification that does not violate the spirit and scope of the claims are intended to be encompassed within the present invention.

One aspect of the present invention provides a fastening member for a hook-and-loop fastener (also referred to simply as “fastening member” in this disclosure) including a base material layer having a fastening member and a printed layer fixed directly on the base material layer. The fastening member for a hook-and-loop fastener of this disclosure can be used as a fastening member that constitutes at least a portion of a hook-and-loop fastener by various conventionally known fastening means.

For example, the fastening member for a hook-and-loop fastener of this disclosure may be a male member provided with an engaging surface made of a plurality of protrusions. Further, it may also be a female member able to engage with this male member and the like provided with an engaging surface made of a plurality of protrusions. Additionally, the structures of both the male member and the female member may also reside on the same surface of the member. The fastening member for a hook-and-loop fastener of this disclosure may be engaged with another fastening member of the same structure or some other fastening member, and furthermore, may directly engage with a wall, fabric, or the like.

Examples of engaging patterns for the hook-and-loop fastener include a hook-and-loop type, a press type and the like. A hook-and-loop type hook-and-loop fastener may be a pair of a male member that has a hook engaging element (also referred to as “stem body” in this disclosure) constituted by a protrusion protruding in the thickness direction of the hook-and-loop fastener and a female member that has a loop engaging element that the hook engaging element (protrusion) can engage. Alternatively, it may also be a pair of fastening members each having both a loop engaging element and a hook engaging element that is a stem body. In this case, there is no distinction between the male member and the female member. Furthermore, a press type hook-and-loop fastener may be a pair of fastening members of the same or different structure that have hook engaging elements protruding in the thickness direction of the hook-and-loop fastener from the base portion. By making the fastening member and the hook engaging element facing each other and applying pressure thereon, mechanical engaging can be obtained.

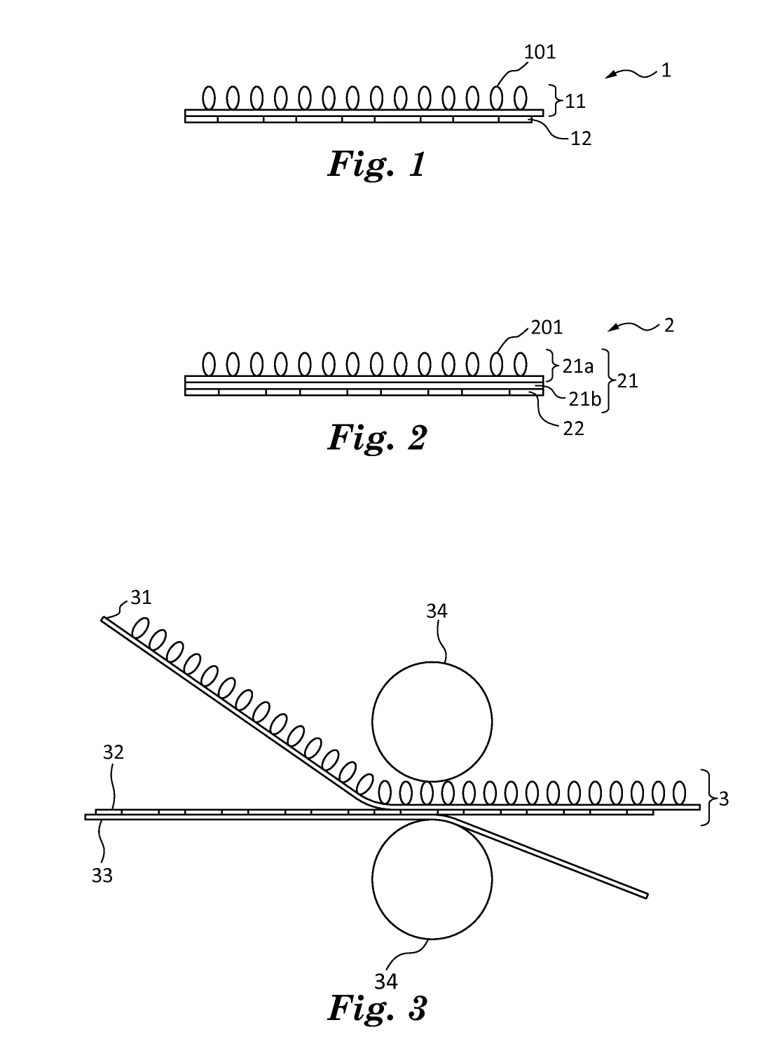

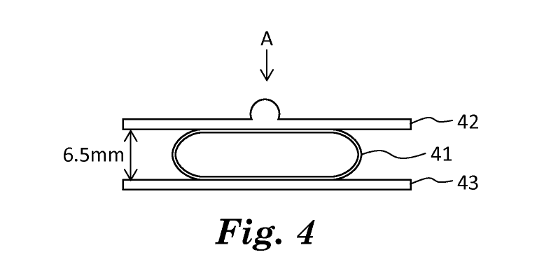

FIGS. 1 and 2 are each diagrams illustrating an example of a fastening member for a hook-and-loop fastener according to an aspect of the present invention. Referring to FIGS. 1 and 2, fastening members 1 and 2 include base material layers 11 and 21, and printed layers 12 and 22 fixed directly on these base material layers. The base material layers 11 and 21 have fastening portions (“loop engaging elements” in FIGS. 1 and 2) 101 and 201, respectively. In this disclosure, the printed layer being directly fixed on the base material layer means that the printed layer is disposed on the base material layer not through another member or layer (i.e., in contact with the base material layer), and that the printed layer is substantially undetachable from the base material layer without its shape deformed. Therefore, a fastening member formed by stacking with the base material layer a printed sheet that has, for example, a printed layer support film and an ink layer provided thereon is distinguished from the fastening member of this disclosure in the fact that the printed layer can usually be detached from the base material layer along with the printed layer support film. In this manner, the shape of the printed layer can be preserved by the base material layer. In other words, the printed layer is supported on the base material layer with the base material layer as a supporting body. Typically, with the fastening member, the printed layer is not fixed to other members such as the printed layer support film, and is fixed only to the base material layer. Further, with the fastening member illustrated in FIGS. 1 and 2, the printed layers 12 and 22 are directly fixed to the base material layers 11 and 21 on surfaces opposing the surfaces that have fastening portions 101 and 201 on the base material layers 11 and 21.

The fastening member of this disclosure has the following advantages compared to a fastening member formed by stacking with the base material layer that has a fastening portion a printed sheet that has, for example, a printed layer support film and an ink layer provided thereon. In other words, with the fastening member of this disclosure, there is no need of a rigid supporting body such as a conventional printed layer support film, because the printed layer is directly fixed on the base material layer. Therefore, reduced breathability and flexibility caused by such a printed layer support film can be avoided, and the fastening member of this disclosure can have favorable breathability and flexibility. These advantages are particularly very advantageous in fitting, for example, when a fastening member is used in a sanitary product. Furthermore, the fastening member of this disclosure does not bring adverse effects in the characteristics of the printed layer.

The fastening member of this disclosure is advantageous in the cost of raw materials and cost of manufacturing because it does not require the use of costly materials (for example, a printed layer support film or the like), and the configuration and the manufacturing method are simple.

The fastening member of this disclosure does not exclude the possibility of having an additional element in addition to the base material layer and the printed layer, but in terms of favorably obtaining the advantages of the fastening member of this disclosure, it is usually preferred that the additional element does not obstruct breathability and flexibility. In these terms, it is generally not desirable to use a general polymer sheet or the like as an additional element. In a typical aspect, the fastening member can be substantially made up of a fastening member and a base material layer.

As a base material layer used in this disclosure, any item generally used in, for example, a hook-and-loop fastener of a sanitary product can be used, and, for example, textiles, woven fabric, non-woven fabric, a resin film having an opening, and the like can be exemplified. For example, a knit, a woven fabric, a non-woven fabric, or a combination of at least two of these (these are favorable because they normally are flexible and breathable) are suitable as a base material layer for a female member, and for example, a resin film having an opening is suitable as a base material layer for a male member. The base material layer may be one layer or may be a plurality of layers.

The base material layer has a fastening portion. The fastening portion can be based on engaging patterns as described above. In other words, the fastening portion may be means of fastening that has a function capable of fastening the obtained fastening member with the same or other fastening member, and various designs of size and shape are possible in accordance with the object. In some aspects, the fastening portion may be a hook engaging element (stem body), a loop engaging element, and the like. Further, in some aspects, when the base material layer is, for example, a non-woven fabric, the fastening portion may be the surface of the non-woven fabric. This is because such a surface can also function as means of fastening that can engage with another type of fastening member such as a hook engaging element (stem body).

For example, the fastening member (male member) having the hook engaging element described above normally has a sheet-shaped base portion, and a plurality of stem bodies arrayed as protrusions on the base portion. The shape of the stem body is not particularly limited, and can be a columnar body such as a cylindrical body, a square column, or a hexagonal column, and furthermore, can have a disk-shaped, mushroom-shaped, hook-shaped, wedge-shaped, arrowhead-shaped or a like-shaped crown portion to improve mechanical engaging work. This is because such a crown portion can more favorably, mechanically engage with a corresponding fastening surface, and can be attached and detached easily.

As for the formation of the fastening member that has a base portion and a stem body, the base material and the stem body may be formed separately, but may also be formed integrally. As a material for the stem body, various thermoplastic resin materials, such as polyethylene (for example, high-density polyethylene), polypropylene, polystyrene, polyvinyl chloride, polyethylene terephthalate, polybutylene terephthalate, nylon, polycarbonate, polymethyl methacrylate, polyacetal, polymethylpentene, acrylonitrile-styrene-butadiene, polyphenylene ether, polyphenylene sulfide, as well as a styrene-based elastomer such as styrene-butadiene-styrene and styrene-isoprene-styrene, an olefin-based elastomer such as an ethylene-α-olefin copolymer, an ester-based elastomer, an amide-based elastomer, an urethane-based elastomer, a vinyl chloride-based elastomer, a silicone-based elastomer, a fluorine-based elastomer, and an alloy thereof.

In favorable aspects, the fastening member of this disclosure is a female member. The female member is normally configured with a knit, a woven fabric or non-woven fabric, or a combination of at least two of these, and has a fastening portion on the surface. This is because the advantage of the flexibility and breathability according to the fastening member of this disclosure is more pronounced in the combination of the base material layer for the female member that normally has flexibility and breathability and the printed layer of this disclosure. In a typical aspect, the fastening portion of the female member is a loop engaging element or a non-woven fabric surface.

In a typical aspect, the base material layer includes a fiber layer. The base material layer may further include an additional layer. Specific examples of an additional layer will be described later. Furthermore, a surface treatment (for example, a corona-discharge treatment, or an E-beam treatment) may be performed on a surface of the printed layer forming surface of the base material layer (for example, the fiber layer when the base material layer is made up of a fiber layer). Furthermore, a treatment such as embossing, coloring, or the like may be performed on the base material layer. The fiber layer can be a three reed knit, a two reed knit, a weft insertion knit, a russell knit or other knits, a jacquard weaving or other woven fabric, a non-woven fabric, and the like.

For example, a non-woven fabric may be manufactured by a carded method, an air-laid method, a spun lace method, a spun bonding method, a melt blowing method, or a combination thereof. Generally, a spun bonded fiber is obtained by extruding a melted thermoplastic polymer from a plurality of thin, normally circular, capillaries of the spinneret as filaments and by rapidly reducing the diameters of the extruded filaments. The spun bonded fiber generally has a small fiber diameter. A meltblown fiber is generally formed by extruding a melted thermoplastic material through a plurality of thin, normally circular, die capillaries to a high speed, normally heated, gas (for example, air) stream as melting yarn or filaments and by thinning the filaments of the melted thermoplastic material to reduce the diameters thereof. Afterward, the meltblown fiber is carried by a high speed air current and is placed on a recovery face, forming randomly strewn meltblown fiber web.

A fiber having a core-sheath structure (composite fiber) can be used as the fiber constituting the non-woven fabric.

In this case, the non-woven fabric may be made up of only a fiber having a core-sheath structure, or may be made up of a fiber having a core-sheath structure and a fiber not having a core-sheath structure. In the fiber having the core-sheath structure, a component contained in the sheath is generally easy to melt compared to a component contained in the core. For example, a fiber having a core-sheath structure with polyethylene and polypropylene, a fiber having a core-sheath structure with polypropylene and modified polyethylene, and a fiber having a core-sheath structure with polypropylene and modified polypropylene are known. In one aspect, in terms of lightweight, high strength, high flexibility, and the like, a non-woven fabric having a core-sheath structure wherein polypropylene is used as a component of the core and polyethylene is used as a component of the sheath is used.

In some aspects, the fiber layer can be a layer having warp yarn and weft yarn, and loop yarn protruding from at least one surface. The warp yarn and weft yarn constitute the base material layer for attaching the fastening member to an application portion (for example, a cloth of a fastening portion of a sanitary product) of a target article (for example, a sanitary product). Further, the loop yarn constitutes the loop engaging element that is the fastening portion. The warp yarn and weft yarn can be spaced at intervals. In this case, even when ink is applied at intersections of the warp yarn and weft yarn so that the ink passes the base material layer in the thickness direction, these intersections give a path for air and the breathability of the fastening member can be favorably maintained. The warp yarn and weft yarn cross each other by being braided, for example.

Each of the warp yarn, weft yarn and loop yarn may be one filament, or may be a bundle of a plurality of filaments. It may be more favorable for the loop yarn to use multifilament rather than monofilament to increase engaging probability with the male member. In this case, there are times when the filament of loop yarn breaks while engaging with the male member when it is excessively thin, and therefore, a filament with a suitable thickness is selected based on the shape and the like of the hook-and-loop fastener. Generally, the total fineness of the filament of warp yarn, weft yarn and loop yarn can be about 20 to 220 dtex, or about 20 to 100 dtex.

Note that the fiber layer may include a yarn other than warp yarn, weft yarn and loop yarn, and, for example, may include a yarn that crosses the warp yarn and weft yarn. Loop yarn may protrude from at least one surface, and may also protrude from both surfaces of the fiber layer. Loop yarn may be knitted into either warp yarn or weft yarn, and may also be knitted into both warp yarn and weft yarn.

In some aspects, the base material layer can include a fiber layer having a first main surface with a loop engaging element and a second main surface opposing the first main surface as the fiber layer. In some aspects, the printed layer is applied to the second main surface, and the second main surface becomes a printed layer forming surface. Here, because the second main surface is relatively smooth when the second main surface is constituted of straight yarn, a clearer display of the design is possible by applying a printed layer to the second main surface.

In a preferred aspect, a loop/straight yarn fiber layer is a knitted fabric constituted of these three yarns: warp yarn, weft yarn (these are each straight yarns), and loop yarn. By this, the first main surface having a loop engaging element and the second main surface constituted of straight yarn are formed. In such a fiber layer, even when ink is applied at the intersections of the three yarns so that the ink passes the base material layer in the thickness direction, these intersections give a path for air and the breathability of the fastening member can be favorably maintained. The knitted fabric described above can be knitted by conventional well-known methods, and can be knitted using a conventional three reed or four reed knitting machine used widely.

The type of fiber constituting the fiber layer can be one type or two or more types, and can be designed based on desired characteristics of the fiber layer. Furthermore, the type of the fiber that constitutes, for example, each yarn of the warp yarn, weft yarn, loop yarn, and the like can be one type or two or more types. As the material of the fiber layer, for example, polyolefin (for example, polyethylene, polypropylene, and the like), polyester, polyamide, polyurethane, ethylene vinyl acetate (EVA), polylactic acid, rayon, a copolymer and mixture thereof, natural fiber, and the like are given. In an aspect with a female member, strong polyamide is used in terms of avoiding the breaking of the female member by engaging with the male member. Furthermore, from consideration of material cost and environmental stability, polyester may be used.

As an additional layer that the base material layer can further include, an adhesive layer, a resin film, a knit, a woven fabric, a non-woven fabric, paper, a stacked body thereof, and the like are given. However, in terms of the breathability of the fastening member, it is preferable that the additional layer not be a resin film. The method for forming an additional layer is not particularly limited, but conventional well-known methods such as coating, dry lamination, extrusion lamination, wet lamination, heat lamination, and ultrasonic may be used.

Further, in a preferable aspect, the base material layer includes an adhesive layer as an additional layer. In this case, the printed layer is adhered to the adhesive layer, and the adhesive layer becomes a printed layer forming surface. Referring to FIG. 2, a base material layer 21 can include a fiber layer 21a and an adhesive layer 21b. When a printed layer 22 is adhered to the base material layer 21 on the adhesive layer 21b, the fixing ability of the printed layer is particularly favorable and advantageous. As the material for the adhesive layer, an adhesive polymer such as acrylic polymer (for example, SK Dyne (an acrylic adhesive commercially available from Soken Chemical), a silicon-based polymer, and a rubber-based polymer, and, for example, a hot-melt adhesive such as Jet-meltTMEC-3784 (commercially available from Sumitomo 3M) can be exemplified. Additives such as a tackifier resin and a cross-linking agent may be additionally optionally combined with the adhesive polymer described above. The thickness of the adhesive layer can be normally about 5 to 200 μm. The adhesive layer can be formed, for example, by drying a material as exemplified above after applying the material onto the surface of the fiber layer of the base material layer.

In addition to this, the base material layer used in the fastening member of this disclosure may be of varieties of conventionally well-known layers. For example, the knitted fabric on the female member described in Japanese Unexamined Patent Application Publication No. 2010-63633A, in other words the loop yarn forms a pile of knitted fabric, the warp yarn and weft yarn form a knitted fabric, and the loop yarn may be a knitted fabric alternately protruding left and right with respect to the warp yarn from only one surface of the knitted fabric. Here, “the loop yarn alternately protrudes left and right with respect to the warp yarn from only one surface of the knitted fabric” means that the loop yarn is formed alternating on the left and right direction on the knitted fabric while maintaining a fixed angle starting from the engaging portion of the warp yarn portion with respect to the surface of the knitted fabric when viewing a cross section of a direction perpendicular to the warp yarn direction. A female member having such a knitted fabric has the advantage of little left and right difference of the engaging force with the male member when engaging with the male member. A hook-and-loop fastener with a small left and right difference in engaging force, is particularly advantageous in applications for a paper diaper. In other words, with a paper diaper, a left and right pair of male members is provided on both sides of the wearer's back side and a left and right pair of female members is provided on the front section of the abdomen. Therefore, at left and right two positions of a paper diaper, the male member and the female member are engaged with each other, but when the left and right difference in engaging force, or in other words, the difference between the peeling force in the left direction and the peeling force in the right direction becomes larger, the wearer may feel uneasy about the sure fixing function of the paper diaper by the hook-and-loop fastener. The female member described above is suitable that because the female member has a structure in which the loop yarn alternately protrudes left and right with respect to the warp yarn, the left and right difference in the engaging force with the male member is small.

Relating to another example of a base material layer, Japanese Unexamined Patent Application Publication No. 2003-204809A describes a hook-and-loop fastener that performs a mechanical coupling by the entangling, meshing, or the like of a pair of fastening portions, wherein at least one fastening portion is made up of a sheet-shaped base material and a plurality of stem bodies arrayed as protrusions on the base material, and each stem body is made up of a compound of a flexible elastic resin material and a highly rigid hard resin material. Such a hook-and-loop fastener has the advantages of being easily manufactured, having excellent strength, having necessary engaging force, and being able to withstand repetitive uses.

In a preferable aspect, the base material layer can have a basis weight of about 10 to 100 g per square meter. When the basis weight is about 10 g/m² or higher, it is easy to retain the shape of the fastening member, and when the basis weight is about 100 g/m² or lower, the rigidity of the fastening member does not become too large and the flexibility of the fastening member is favorable.

The printed layer has at least an ink layer and is one layer or a plurality of layers. The printed layer may be constituted of only an ink layer, or may have a base coat or a top coat in addition to the ink layer. The base coat and the top coat each contributes to the improvement of the fixing ability of the ink layer on the base material layer.

The ink layer and the optional base coat and top coat can each be a continuous layer on the base material layer, may be disposed discontinuously, and can be appropriately designed based on a desired object, for example, the target design of the ink layer. A design including a character, a picture, a pattern, and the like can be optionally selected.

As the material of the ink layer, various conventional well-known inks are given and water soluble inks and solvent inks can be used. As resin contained in the ink, conventionally normally used resin can be used. For example, acrylic resin, polyurethane resin, polyamide resin, urea resin, polyester resin, vinyl chloride resin, vinylidene chloride resin, vinyl chloride-vinyl acetate copolymer resin, ethylene-vinyl acetate copolymer resin, olefin resin, chlorinated olefin resin, epoxy resin, petroleum-based resin, cellulose derivative resin, and the like are used. With an ink layer with excellent toughness, the ink layer is less likely to drop off even when the ink layer is exposed during the use of an article having the fastening member of this disclosure. In this light, for example, acrylic ink, urethane-based ink, or the like is suitable.

It is preferable that the ink layer itself have adhesiveness in terms of the fixing ability of the printed layer on the base material layer. As the material for forming an ink layer having adhesiveness, a mixture of ink and the material used in the adhesive layer described above can be exemplified.

In terms of toughness of the printed layer and wearing feeling (flexibility, breathability, and the like) when the printed layer is used in a sanitary product, the thickness of each layer constituting the printed layer can be, but is not limited to, for example, about 0.5 μm or more to 20 μm or less, about 1 μm or more to 15 μm or less, or about 2 μm or more to 10 μm or less.

A manufacturing method for the fastening member for a hook-and-loop fastener of this disclosure includes forming the printed layer on the base material layer. As a method for directly fixing the printed layer on the base material layer, for example, a method for coating the material for forming the printed layer (also referred to as printed material in this disclosure) on the printed layer forming surface of the base material, a method for transferring a printed layer previously formed on a liner onto the base material layer and removing the liner such as described below, and the like can be used. The method for transferring described above is preferable in that it is difficult for the ink of the printed layer to penetrate the base material layer.

As one aspect of the present invention,

a method for manufacturing a fastening member for a hook-and-loop fastener including a printed layer and a base material layer having a fastening portion is provided. The method comprises the steps of:

forming a printed layer on a liner,

transferring the printed layer to a base material layer having a fastening portion, and

removing the liner from the printed layer.

The method will be described below.

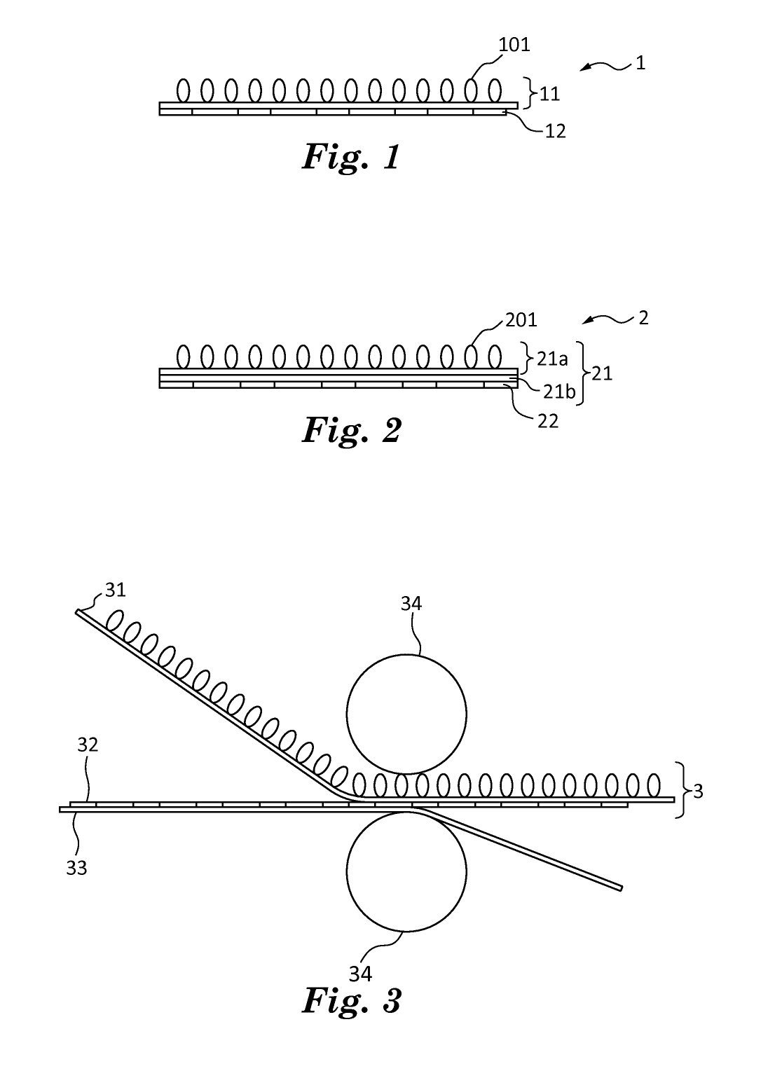

FIG. 3 is a diagram illustrating an example of a method for manufacturing a fastening member for a hook-and-loop fastener according to an aspect of the present invention. First, referring to FIG. 3, a printed layer 32 is formed on a liner 33. It is preferable that the surface of the liner have adequate releasability so as to transfer the printed layer to the base material layer. For the liner, a variety of conventionally well-known sheets can be used as the transfer liner. For example, silicone-coated kraft paper, silicone-coated polyethylene-coated paper, a polymer material (for example, polyethylene, polypropylene, and the like) coated or not coated with silicone, a polymeric release agent, a base material coated with, for example, silicone urea, urethanes, long chain alkyl acrylate and the like, and the like are given. As appropriate release liners that are commercially available, there are release liners sold under the product name of “POLYSLIK” by the Rexam Release of Oakbrook, Illinois and “EXHERE” by the P.H Glatfelter Company of Spring Grove, Pa. The printed layer forming surface of the liner may be applied with, for example, emboss processing or the like.

The printed material is applied on such a liner. In a typical aspect, the ink is applied on the liner by any printing method such as roll coating, gravure coating, curtain coating, spray coating, screen printing, and the like. Alternatively, for example, a top coat, ink, and a base coat may be applied in this order on the liner. With the procedures described above, a stacked body is obtained by the formation of the printed layer on the liner.

Next, the printed layer 32 is transferred from a liner 33 to a base material layer 31 by passing this stacked body and the base material layer 31 between a pair of rollers 34. By this, a fastening member 3 with the printed layer 32 formed on the base material layer 31 can be obtained. Here, the printed layer 32 is formed on a surface opposing the surface having the fastening portion on the base material layer.

Note that when the printed layer has an ink layer and a base coat and/or a top coat, the printed layer may be sequentially formed in a plurality of processes. For example, with a printed layer made up of a base coat, an ink layer and a top coat, the base coat may be formed on the base material layer, the ink layer may be transferred, and then the top coat may be formed. The printed layer may be directly fixed to the base material layer, and the method for forming the printed layer is not limited.

In a preferable aspect, the fastening member is soft and flexible. For example, when the base material layer has a fiber layer having warp yarn and weft yarn, it is preferable that the rigidity of the fastening member be about 0.8 N/inch or more to 3.5 N/inch or less in an MD direction (warp yarn direction), and about 0.3 N/inch or more to 3.5 N/inch or less in a CD direction (weft yarn direction) that forms an angle of 90° with respect to the MD direction described above. When the rigidity is within the range described above, the fastening member can have excellent flexibility while still having favorable mechanical strength. The rigidity described above is the value measured by the method described in the EXAMPLES section of this disclosure.

In a preferable aspect, the fastening member has excellent breathability. More specifically, it is preferable that the air permeability of the fastening member measured by the Gurley method be about 5 seconds or less in terms of giving a good wearing feeling for the sanitary product. It is more preferable that the air permeability be about 3 seconds or less, and further preferable that the air permeability be about 1 second or less. The lower limit is not particularly limited. However, in one aspect, the lower limit is about 0.1 seconds or more.

The fastening member of this disclosure can be used for fixing various application objects for various articles such as flooring, a wall material, clothing, a cleaning member, and an automotive material. However, the fastening member of this disclosure is particularly suitable as a fastening member of a sanitary product such as a paper diaper, a sanitary napkin, and a breast pad because the fastening member can have favorable flexibility and breathability thanks to the constitution.

Another aspect of the present invention provides a sanitary product that includes a hook-and-loop fastener including a fastening member for a hook-and-loop fastener according to an aspect of the present invention described above.

As a sanitary product, a diaper for children and adults, a sanitary napkin and a napkin for other uses, and the like are given. In a typical aspect, the sanitary product is a diaper. In a typical aspect, the female member (or male member) that is the fastening member of this disclosure can be used as a hook-and-loop fastener for a sanitary product, or more preferably a diaper, in combination with the male member (or female member) that is the fastening member of this disclosure or any other article.

In a preferable aspect, the sanitary product has excellent breathability. More specifically, it is preferable that the air permeability of the sanitary product measured by the Gurley method be about 5 seconds or less in terms of giving a good wearing feeling for the sanitary product. It is more preferable that the air permeability be about 3 seconds or less, and further preferable that the air permeability be about 1 second or less. The lower limit is not particularly limited. However, in one aspect, the lower limit is about 0.1 seconds or more.

The method for manufacturing a sanitary product is not particularly limited. However, for example, the method below can be exemplified. As elements other than the fastening member on the sanitary product, any conventionally well-known element can be adopted, and details will not be described herein. With a sanitary product, the method for attaching the fastening member to an application portion can be any conventionally well-known method. The printed layer forming surface of the fastening member is joined to the application portion by a conventionally well-known joining method (adhesion by glue, thermal fusion bonding, and ultrasonic machining and the like, sewing, mechanical fixing by a stapler and the like). For fastening by means of gluing, known adhesives such as rubber based adhesives such as SIS and SBS, acryl based adhesives, silicone based adhesives, EVA based adhesives, and the like may be suitably selected as required, but the adhesive is not limited thereto.

### EXAMPLES

The present invention will now be described in further detail using examples, but the present invention is not limited to these examples.

### Working Example

Using the materials described below, a female member for a hook-and-loop fastener which is an example of the fastening member of this disclosure was produced.

Fiber layer: A warp knitted fabric having a loop engaging element (material: nylon, basis weight: 21 g/m²)

Adhesive: An urethane adhesive (commercially available from Mitsui Chemicals Co., Ltd. as “PU A969v/A5”)

Liner: A biaxially oriented polypropylene release-treated by a silicone coating (commercially available from Shuangliang Inc.)

Ink: Univure Ink (commercially available from Dainippon Printing Ink Manufacturing Co., Ltd.)

Ink was applied to the release treated surface of the liner by gravure coating, and a transfer film made by forming the printed layer on the liner was obtained. The basis weight of the printed layer was 3 g/m². Furthermore, 5 g/m² of adhesive was gravure coated to the surface having no loop of the fiber layer, and, after drying, the adhesive layer was formed. Next, the printed layer was transferred to the base material layer by causing the adhesive layer of the base material layer and the printed layer of the transfer film to come into contact with each other using a laminator (manufactured by FUJI KIKAI KOGYO Co., Ltd.) and to pass between the pair of rollers. The obtained stacked body constituted of the base material layer and the printed layer was stored at 35° C. for 24 hours so as to allow the adhesive and printed layer to be hardened. Afterward, the liner was detached from the printed layer. The fastening member was obtained with the procedures described above. The printing properties (clearness, fixing ability) of the obtained fastening member were favorable.

### Comparative Example

Using the materials described below, a female member for a hook-and-loop fastener was produced.

Fiber layer: A warp knitted fabric having a loop engaging element (material: nylon, basis weight: 21 g/m²)

Support film: A biaxially oriented polypropylene film with one surface corona-discharge treated (basis weight: 10 g/m²)

Ink: Univure Ink (commercially available from Dainippon Printing Ink Manufacturing Co., Ltd.)

Ink was applied to the corona-discharge treated surface of the support film by gravure coating, and a printed film made by forming the printed layer on the support film was obtained. The basis weight of the printed layer was 3 g/m². Furthermore, 5 g/m² of adhesive was gravure coated to the surface having no loop of the fiber layer, and, after drying, the adhesive layer was formed. Next, a stacked body constituted of the base material layer and the print film was obtained by causing the adhesive layer of the base material layer and the printed layer of the print film to come into contact with each other using a laminator (manufactured by FUJI KIKAI KOGYO Co., Ltd.) and to pass between the pair of rollers. The obtained stacked body was stored at 35° C. for 24 hours so as to allow the adhesive and printed layer to be hardened. The fastening member was obtained with the procedures described above. With this fastening member, the printed layer is adhered with the base material film with the printed layer fixed on the biaxially oriented polypropylene film that is the support film.

### Reference Example

A non-woven fabric (loop) female member having no printed layer (manufactured by 3M Inc. fiber: core-sheath structure fiber constituted of core (polypropylene)/sheath (polyethylene), basis weight: 43 g/m²) was used as a sample of the fastening member.

### Performance Evaluation

The performance of the fastening member were evaluated as described below. The results are shown in Table 1. Note that below, the MC direction and the CD direction in the working example and the comparative example are the warp yarn direction and the weft yarn direction of the fiber layer of the base material layer, respectively, and in the reference example, the longitudinal direction when the non-woven fabric is being manufactured is the MD direction and the width direction when the non-woven fabric is being manufactured is the CD direction.

### Air Permeability (Gurley Method)

The moisture permeability of a sample having a size of MD direction 50 mm×CD direction 50 mm which was cut out from the fastening member, using a moisture permeability tester (manufactured by Toyo Seiki Seisaku-sho, Ltd., Gurley Densometer).

### Rigidity of the Fastening Member]

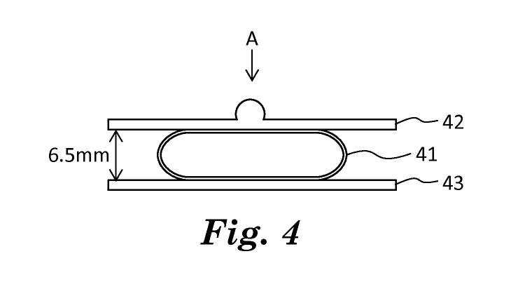

FIG. 4 is a diagram describing a method for measuring the rigidity of the fastening member in the working example. A tape sample 41 having a size of MD direction 300 mm×CD direction 25 mm was cut out from the fastening member. Next, both end portions in the MD direction of the obtained tape sample 41 are overlapped with each other so that the tape sample 41 is formed into an annular shape, and the tape sample 41 is interposed between a compression board 42 and a sample holder 43. The resilience was measured when pressing, in the direction of arrow A, the tape sample 41 by a loop pressing distance of 6.5 mm at a pressing speed of 3.5 mm/second using a Tensilon universal tester (Toyo Seiki Seisaku-sho, Ltd., loop stiffness tester), and the peak value of the resilience was recorded.

### 90° Peeling Strength and Shear Strength of the Fastening Member

A sample having a size of MD direction 25 mm×CD direction 20 mm was cut out from the fastening member as a female member and pasted on a stainless panel.

A sample having a size of MD direction 25 mm×CD direction 20 mm was cut out from the hook-and-loop fastener male member (manufactured by Sumitomo 3M Ltd., NC-2050) and was pasted on a support tape.

The 90° peeling strength was measured by engaging the female member and the male member after aligning the MD directions thereof, and performing a 90° peel test in the MD direction at a tensile speed of 300 mm/minute using a Tensilon universal tester (manufactured by SHIMADZU CORPORATION, precision universal tester AUTOGRAPH AG-X).

Furthermore, the shear strength was measured by that a sample having a size of MD direction 50 mm×CD direction 10 mm was cut out from the fastening member (female member), the above-described male member having a size of more than MD direction 25 mm×CD direction 90 mm was cut out, the both were engaged with each other after aligning the CD directions thereof, and a shear peel test in the CD direction was performed at a tensile speed of 30 mm/minute, using the Tensilon universal tester described above.

TABLE 1

| | | Fastening Member Evaluation | | | |
|---|---|---|---|---|---|
| Example | Fastening Member | | Air Permeability | Rigidity (N) | |
| Number | Constitution | Measurement N | (second/50 cc) | MD | CD |
| Working | Fiber | 1 | 0.4 | 1.125 | 0.45 |
| Example | Layer/Adhesive | 2 | 0.9 | 1.1 | 0.37 |
| | Layer/Printed | | | | |
| | Layer | | | | |
| Comparative | Fiber | 1 | 25.6 | 4.2 | 3.7 |
| Example | Layer/Adhesive | 2 | 19.4 | 4.2 | 3.6 |
| | Layer/Print Film | | | | |
| | (Ink Layer/Support | | | | |
| | Film) | | | | |
| Reference | Non-woven Fabric | 1 | 0.1 | 4.25 | 1.26 |
| Example | Female Member | 2 | 0.4 | 4.7 | 1.4 |
| | | Fastening Member Evaluation | | | |
| | | | | 90° Peeling | Shear |
| Example | Fastening Member | Basis Weight | Thickness | Strength | Strength |
| Number | Constitution | (g/m²) | (μm) | (N) | (N) |
| Working | Fiber | 0.0848 | 138.0 | 2.332 | 111.275 |
| Example | Layer/Adhesive | 0.085 | 142.8 | 3.1699 | 119.431 |
| | Layer/Printed | | | | |
| | Layer | | | | |
| Comparative | Fiber | 0.0967 | 168.0 | 1.0015 | 80.8937 |
| Example | Layer/Adhesive | 0.0954 | 162.4 | 1.2325 | 67.2688 |
| | Layer/Print Film | | | | |
| | (Ink Layer/Support | | | | |
| | Film) | | | | |
| Reference | Non-woven Fabric | 0.1105 | 208.0 | 2.0762 | 42.8563 |
| Example | Female Member | 0.1088 | 178.0 | 2.0134 | 42.6625 |

In the working example where the printed layer is fixed directly on the base material layer, the rigidity was kept low; thus, the flexibility was favorable. Furthermore, in the working example, breathability is notably excellent compared to the comparative example where the printed layer is provided on the biaxially oriented polypropylene film, and there is no large reduction in breathability compared to the reference example not having a printed layer, and favorable breathability is thereby realized while still having a printed layer.

The fastening member for a hook-and-loop fastener of this disclosure is suitably applicable for a sanitary product and the like, such as a diaper for children and adults, a sanitary napkin and a napkin for other uses, and the like.

1. A fastening member for a hook-and-loop fastener comprising:
a base material layer having a fastening portion, and
a printed layer fixed directly on the base material layer.

2. The fastening member for a hook-and-loop fastener according to claim 1, wherein the fastening member is a female member.

3. The fastening member for a hook-and-loop fastener according to claim 2, wherein the base material layer comprises a fiber layer having a first main surface having a loop engaging element on a surface thereof, and a second main surface opposing the first main surface, and
the printed layer is fixed directly on the second main surface.

4. The fastening member for a hook-and-loop fastener according to claim 1, wherein the base material layer comprises an adhesive layer and the printed layer is fixed directly on the adhesive layer.

5. A method for manufacturing a fastening member for a hook-and-loop fastener including a printed layer and a base material layer having a fastening portion, comprising the steps of:
forming a printed layer on a liner,
transferring the printed layer to a base material layer having a fastening portion, and
removing the liner from the printed layer.

6. A sanitary product comprising the fastening member for a hook-and-loop fastener of claim 1.

7. The sanitary product according to claim 6, wherein the sanitary product is a diaper.
